(19) United States
(12) Patent Application Publication
(10) Pub. No.: US 2015/0224331 A1
(43) Pub. Date: Aug. 13, 2015

(54) SPINAL CORD PULSATION-CANCELATION INJECTION SYSTEM

(71) Applicant: THE REGENTS OF THE UNIVERSITY OF CALIFORNIA, Oakland, CA (US)

(72) Inventor: Martin Marsala, Solana Beach, CA (US)

(73) Assignee: The Regents Of The University Of California, Oakland, CA (US)

(21) Appl. No.: 14/430,384

(22) PCT Filed: Sep. 23, 2013
(86) PCT No.: PCT/US13/61144

§ 371 Date: Mar. 23, 2015

Related U.S. Application Data
(60) Provisional application No. 61/704,959, filed on Sep. 24, 2012.

(51) Int. Cl. A61N 2/00; A61M 5/32

(52) U.S. Cl. CPC A61N 2/002; A61M 5/32; A61M 2202/09; A61M 2202/206; A61M 2210/1003

(57) ABSTRACT

Delivery devices, systems, and methods related thereto may be used in humans for spinal delivery of cells, drugs or vectors. The patient population may include patients with spinal traumatic injury, amyotrophic lateral sclerosis, multiple sclerosis, spinal ischemia and any other spinal neurodegenerative disorders which will require spinal cell, vector or drug delivery. The delivery device compensates for spinal cord pulsation during such injections.

### CROSS REFERENCE TO RELATED APPLICATION(S)

This application is a 35 U.S.C. §371 National Stage of International Application No. PCT/US2013/061144 filed on Sep. 23, 2013, and claims the benefit of priority under 35 U.S.C. §119(e) of U.S. Ser. No. 61/704,959, filed Sep. 24, 2013, the entire content each of which is incorporated herein by reference in their entirety for all purposes.

### GRANT INFORMATION

This invention was made with government support under Grant No. NS051644-02A2 awarded by the National Institutes of Health. The United States government has certain rights in the invention.

### BACKGROUND OF THE INVENTION

1. Field of the Invention

The invention relates generally to a drug or cell delivery system and more specifically, to a drug or cell delivery system that eliminates spinal cord pulsation effects during spinal cord injections in large animal species and humans.

2. Background Information

The spinal cord is a delicate structure that rests within the spinal canal and is surrounded by a tough outer covering, called the dura. Normally, the spinal cord ends at about the first or second lumbar vertebrea in the adult. The spinal canal is surrounded and protected by the bony structure of the spinal column (or vertebrea). Cerebrospinal fluid (CSF) surrounds the spinal cord and flows from the brain, down the spinal canal and back up to the brain. Many nerves originate from the spinal cord, and are responsible for movement and sensation of the arms, legs and torso.

Intraspinal injections have been used for spinal anaesthesia, chemotherapy, pain management applications, and for taking samples of cerebral spinal fluid. Administering a substance to the spaces or potential spaces surrounding the spinal cord is often performed in order to avoid the blood-brain barrier. Intraspinal grafting of human neural stem cells represents a promising approach to promote recovery of function after spinal trauma.

Dorso-ventral spinal cord pulsation resulting from respiration and/or cerebrospinal fluid movement represents a serious risk factor during the procedure of direct spinal parenchymal injections. Currently existing devises use internally mounted frames that are places over laminectomy sites and use freely floating cannulas that are advanced into the spinal cord parenchyma while being firmly attached to the manipulator. The cannulas are then released from the holder to create a “free-floating” effect. However, such devices are complicated to use due to the need for repetitive immobilization and release of the floating cannula in the Z-arm of the injector between individual injections.

### SUMMARY OF THE INVENTION

The present invention is based on the utilization of repulsive forces produced by micromagnets to create a spring effect that compensates for spinal cord pulsation during intraspinal injections.

Accordingly, in one aspect, there is provided a spinal cord pulsation-cancelation injection device. The device includes a frame having an elongated body and a plurality of holders extending therefrom; a plurality of first magnets, each being fixedly attached to a holder; a tube having a first end and a second end, the tube being slidingly disposed within through-holes disposed in each holder and in each first magnet; a plurality of second magnets fixedly attached to an exterior surface of the tube; and a needle fixedly attached to the first end of the tube. The frame may be made from any non-corrosive metal, such as stainless steel. The needle may range from about 27 to about 32 gauge. Each of the first magnets and the each of the second magnets may be disposed such that a north pole of one first magnet faces a north pole of one second magnet or a south pole of one first magnet faces a south pole of one second magnet, thereby providing a magnetic repulsive force upon which the tube floats.

In various embodiments, the frame comprises two holders, each having attached thereto a first magnet, and a single second magnet is fixedly attached to the tube. In other embodiments, the frame comprises two holders, each having attached thereto a first magnet, and two second magnets are fixedly attached to the tube.

The device may further include a stop ring fixedly attached to the first end of the tube or an area near the first end of the tube. The device may further include a stop ring fixedly attached to the second end of the tube or an area near the second end of the tube. The device may further include tubing removably attached to the second end of the tube and configured for supplying a substrate to the needle.

In another aspect, there is provided a spinal cord pulsation-cancelation injection system. The system includes the spinal cord pulsation-cancelation injection device described herein and a reservoir in fluid communication with the needle, the reservoir containing a substrate to be administered to a subject. The system may further include a digital microinjector configured to control flow of the substrate through the needle. In various embodiments, the substrate is selected from the group consisting of cells, drugs, viruses, plasmids, and growth factors.

In various embodiments, the frame comprises two holders, each having attached thereto a first magnet, and a single second magnet is fixedly attached to the tube. In other embodiments, the frame comprises two holders, each having attached thereto a first magnet, and two second magnets are fixedly attached to the tube.

In yet another aspect, there is provided a method of compensating for spinal cord pulsation during administration of a substrate to a spinal cord of a subject. The method includes positioning the spinal cord pulsation-cancelation injection system described herein over the spinal cord of the subject; lowering the needle into the spinal cord; and delivering a dose of the substrate to the spinal cord, wherein the needle and tube of the device float due to magnetic repulsive forces within the device, thereby compensating for spinal cord pulsation. In various embodiments, the method further includes repeating each of the steps at multiple sites along the spinal cord. The step of delivering may include activating a digital microinjector configured to control flow of the substrate through the needle. The step of lowering the needle may include inserting the needle into the spinal parenchyma until a needle stop ring that is fixedly attached to the needle contacts the subject. Accordingly, the present invention also provides use of the spinal cord pulsation-cancelation injection system described herein to compensate for spinal cord pulsation during administration of a substrate.

### BRIEF DESCRIPTION OF THE DRAWINGS

FIG. 1 is pictorial diagram showing a first exemplary embodiment of the spinal cord pulsation-cancelation injection system.

FIG. 2 is pictorial diagram showing a second exemplary embodiment of the spinal cord pulsation-cancelation injection system.

FIG. 3 is pictorial diagram showing use of the spinal cord pulsation-cancelation injection system for delivering a cell suspension to the spinal cord of a subject.

FIG. 4 is a flow chart describing steps for delivering a cell suspension to the spinal cord of a subject using the spinal cord pulsation-cancelation injection system.

### DETAILED DESCRIPTION OF THE INVENTION

The present invention is based on the utilization of magnetic repulsive forces to create a spring effect that compensates for spinal cord pulsation during intraspinal injections. Once the injection needle is advanced in the spinal parenchyma, it can then fluctuate with any pulsation of the spinal cord in the dorso-ventral direction due to the magnetic repulsive forces acting on the needle holder. As such, the present invention provides a spinal cord pulsation-cancelation injection system that may be used for spinal cord cell and vector delivery in large animals and humans.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “dorsoventral” or “dorso-ventral” is an adjective that refers to extending along or denoting an axis joining the dorsal and ventral surfaces of a primate. Included in the term is extending from the back to the belly of the animal.

As used herein, the term “pulsation” when used in the context of the spinal cord of a human or animal, refers to involuntary movement of the spinal cord as a result of cardiac pulsation and/or respiration. Recent studies have indicated that spinal cord pulsation is derived mainly from the radicular arteries, rather than from the change in the brain volume or the influence of central and peripheral circulation. However, included in the term is pulsation of the spinal cord as a result of cerebrospinal fluid moving back and forth within the cervical canal.

As used herein, the terms “stereotaxis,” “stereotaxic,” and “stereotactic” are used interchangeably to refer to methods in neurosurgery and neurological research for locating points within the brain or spinal cord using an external, three-dimensional frame of reference usually based on the Cartesian coordinate system. Methods of steretactic surgery are known in the art.

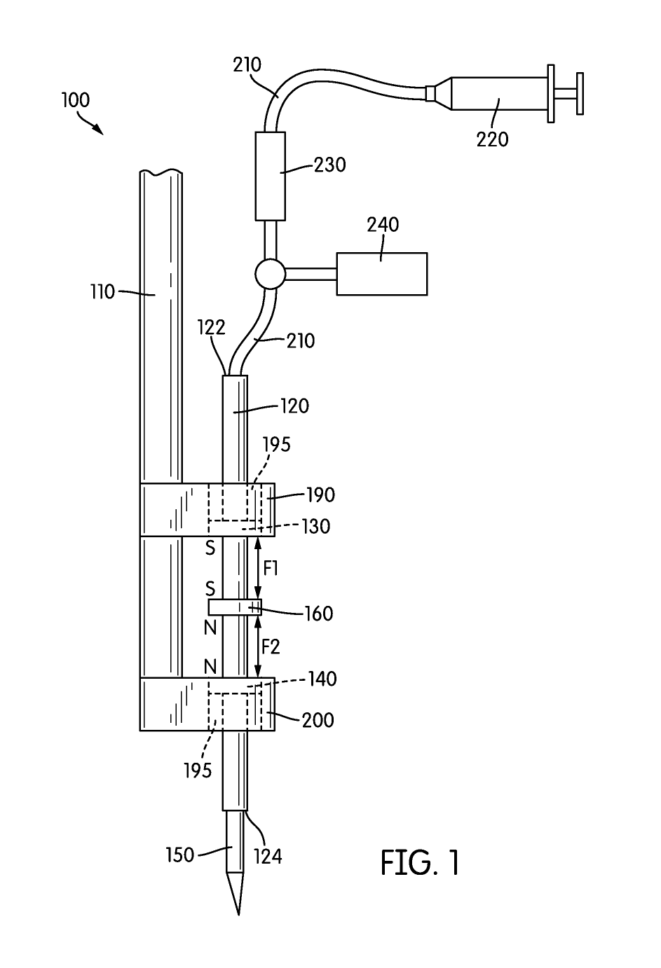

Referring to FIG. 1, an exemplary embodiment of the spinal cord pulsation-cancelation injection system 100 is shown. The system includes a frame 110 having an elongated body and a plurality of holders (190, 200) extending therefrom. The body of the frame may be a circular or square bar having a first end and a second end. The first end is fixedly connected to an XYZ manipulator (not shown), which is mounted directly onto a stereotaxic frame (not shown). While the body may be made from any rigid material, in certain embodiments, the bar is made from any non-corrosive metal, such as stainless steel. Extending from the second end of the bar is a first holder 200 of the plurality of holders. One or more additional holders may extend in the same direction, and parallel to, the first holder 200. In certain embodiments, the frame will include a first holder 200 extending from the second end of the bar and a second holder 190 extending from the bar at a predetermined distance from the first holder 200. The predetermined distance between the first and second holders may range from about 1 cm to about 5 cm (i.e., 1 cm, 2 cm, 3 cm, 4 cm, 5 cm, or any fraction there between). Disposed within each holder is a through-hole 195 such that the through-hole of one holder is aligned with the through-hole of each successive holder.

Slidingly disposed within the through-holes 195 of each holder is a tube 120 having a top end 122 and a bottom end 124. The tube 120 may be formed from the same metal material from which the frame is formed. Thus, in an exemplary embodiment, the tube is made from stainless steel. Fixedly mounted to the bottom end 124 of the tube 120 is an injection needle 150, which may be from about 27 to about 32 gauge, and may be made from stainless steel or other non-corrosive materials. Plastic or Teflon tubing may be removably attached to the top end 122 of the tube for connecting the tube 120 to a reservoir 230 and/or syringe 220 for supplying a substrate to the spinal cord of a subject. In certain embodiments, injections may be performed by using a digital microinjector 240. Optionally disposed between the syringe 220 and the tube 120 of the system may be a cell suspension reservoir 230 to minimize sedimentation of cells in the tubing when mounted vertically to the XYZ manipulator (not shown).

As used herein, the term “substrate” refers to any injectable substance, including but not limited to cells, drugs, viruses, plasmids, growth factors and the like. The substrate may take any suitable form of matter, including a liquid, a suspension, a gel, an encapsulated solid, a nanoparticle suspension, a slow- or extended-release polymer composition and the like.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Disposed in a surface of the first holder 200 and second holder 190 is a ring magnet having a north-south polarity and a through-hole that corresponds to, and is aligned with, the through-hole 195 of each holder. As shown in FIG. 1, a first magnet 140 is disposed in the first holder 200 and a second magnet 130 is disposed in the second holder 190. Fixedly attached to an exterior surface of the tube 120 is one or more ring magnets 160, which may be disposed between a pair of the holders. In the exemplary embodiment shown in FIG. 1, a single ring magnet 160 is fixedly attached to the exterior surface of tube 120 such that the ring magnet 160 is located between each of the first magnet 140 and second magnet 130. The ring magnet 160 also has a north-south polarity, and is disposed on the tube 120 such that the north pole of the ring magnet 160 faces the north pole of the first magnet 140 and the south pole of the ring magnet 160 faces the south pole of the second magnet 130. Of course, the ring magnet 160 may be disposed on the tube 120 such that the north pole of the ring magnet 160 faces the north pole of the second magnet 130 and the south pole of the ring magnet 160 faces the south pole of the first magnet 140. As such, a repulsive magnetic force F1 is created between the ring magnet 160 and the magnet 130 of the second holder 190. Likewise, a similar repulsive magnetic force F2 is created between the ring magnet 160 and the magnet 140 of the first holder 200. With repulsive force F1 being equal to repulsive force F2, tube 120 floats within the through-holes 195, thereby creating a spring effect. Optionally disposed at the top end 122 and the bottom end 124 of the tube 120 is one or more plastic stop rings (not shown) to prevent the tube from exceeding a maximum allowable range of movement.

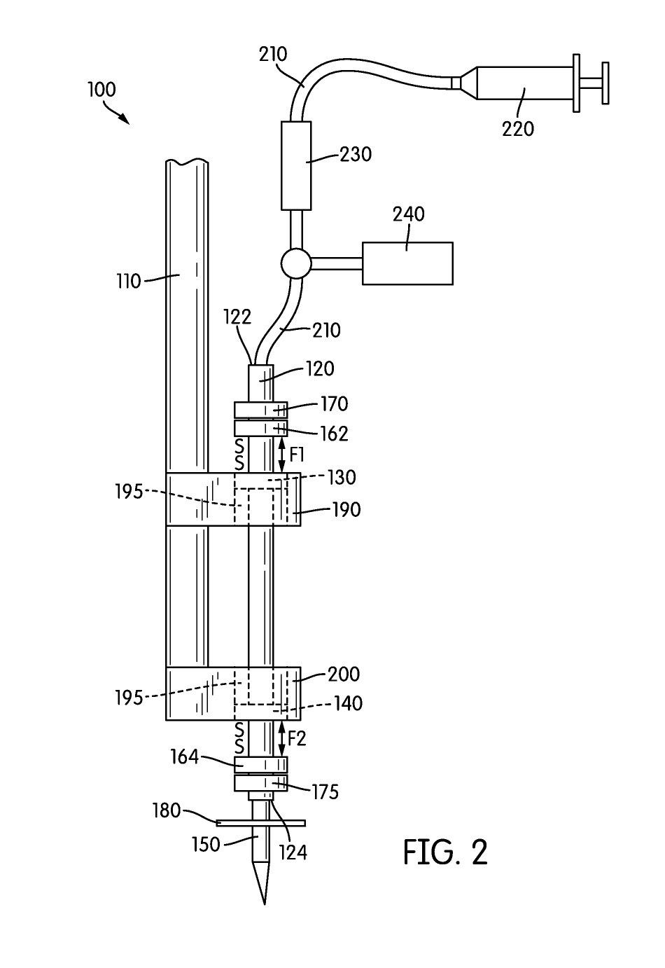

With reference now to FIG. 2, an alternative exemplary embodiment of the spinal cord pulsation-cancelation injection system 100 is shown, wherein like reference numerals refer to like elements throughout. Thus, only the differences between the first exemplary embodiment shown in FIG. 1 and the second exemplary embodiment shown in FIG. 2 will be discussed herein.

As shown in FIG. 2, the system includes a first magnet 140 is disposed in the first holder 200 and a second magnet 130 is disposed in the second holder 190. Fixedly attached to an exterior surface of the tube 120 is one or more ring magnets 160. In the exemplary embodiment shown in FIG. 2, two ring magnets are fixedly attached to the exterior surface of tube 120 at a position that places the ring magnets in close proximity to the magnets of the first holder 200 and second holder 190. Thus, as shown, an upper ring magnet 162 is disposed at the top end 122 or in an area adjacent to the top end 122 of the tube 120. Likewise, a lower ring magnet 164 is disposed at the bottom end 124 or in an area adjacent to the bottom end 124 of the tube 120. Each of the upper magnet 162 and lower magnet 164 has a north-south polarity, and are disposed on the tube 120 such that the south pole of the upper magnet 162 faces the south pole of the second magnet 130 and the south pole of the lower magnet 164 faces the south pole of the first magnet 140. Of course, the upper and lower magnets may be disposed such that the north pole of the upper magnet 162 faces the north pole of the second magnet 130 and the north pole of the lower magnet 164 faces the north pole of the first magnet 140. As such, a repulsive magnetic force F1 is created between the upper magnet 162 and the magnet 130 of the second holder 190 Likewise, a similar repulsive magnetic force F2 is created between the lower magnet 164 and the magnet 140 of the first holder 200. With repulsive force F1 being equal to repulsive force F2, tube 120 floats within the through-holes 195, thereby creating a spring effect. Optionally disposed at the top end 122 and the bottom end 124 of the tube 120 is one or more plastic stop rings (not shown) to prevent the tube from exceeding a maximum allowable range of movement.

In both exemplary embodiments, a sterile needle stop ring 180 may be fixedly attached to the needle 150 to serve a guide to a surgeon as to the maximum distance that the needle 150 will be lowered into the spinal cord during the procedure. When present, the stop ring 180 may be positioned along the length of the needle 150 such that when the needle reaches a predetermined depth, the stop ring 180 contacts the subject/patient.

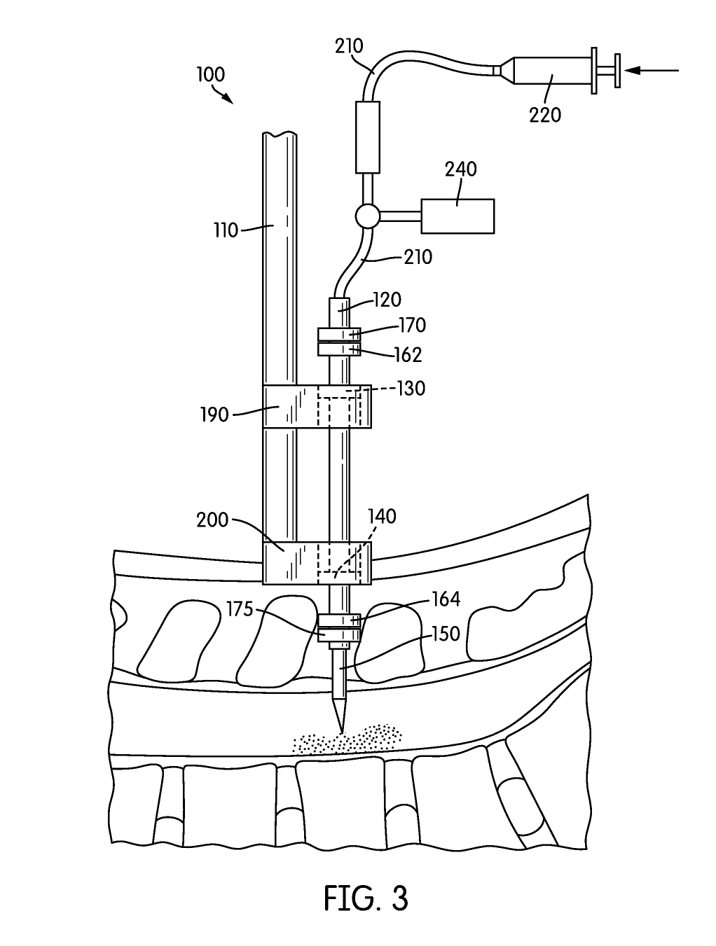

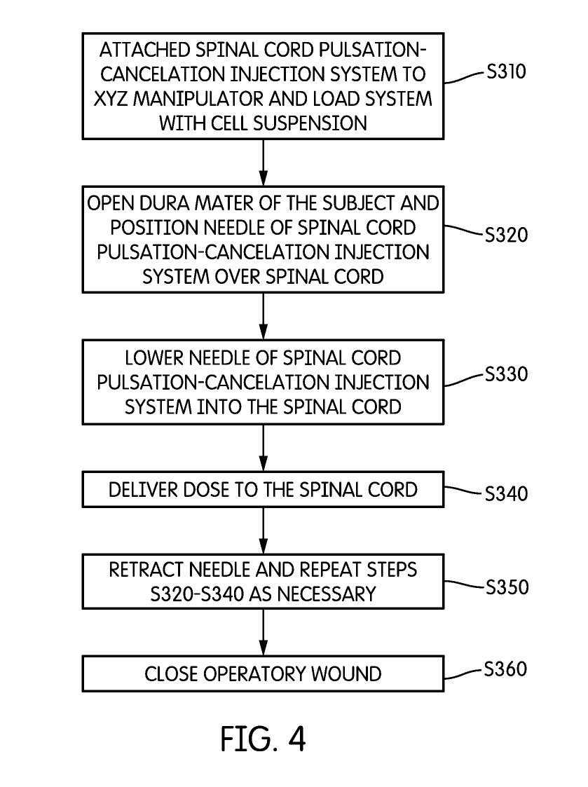

Referring now to FIGS. 3 and 4, use of the system 100 is as follows. The surgical table with stereotactic frame is prepared for the procedure and the subject is positioned on their prone surface. A standard posterior approach is performed, targeting T1 to T10 of the spinal cord, followed by an “open door” laminoplasty, leaving the dura matter intact. Sterile saline may be used to clean/flush the operatory wound, and sterile fields may be applied to protect the subject.

The XYZ manipulator (not shown) is then attached to the stereotactic frame above the operatory wound of the subject. The spinal cord pulsation-cancelation injection system 100 is then attached to the XYZ manipulator (not shown) and connected to a cell suspension reservoir 230 and syringe containing the suspension to be administered via sterile tubing 210. The contents of the syringe are then loaded into the cell suspension reservoir 230 (S310). The sterile tubing is run through a digital microinjector 240, which is operated to remove any air gaps in the lines.

Continue the surgical procedure by opening the dura mater, perform a longitudinal incision in the dura mater, avoiding damage to blood vessels. Position the needle 150 of the spinal cord pulsation-cancelation injection system 100 above the spinal cord at the point of injection (S320).

The needle is then lowered into the spinal cord parenchyma, avoiding damage to blood vessels under visual guidance (S330). The digital microinjector 240 is then activated to deliver the dose to the spinal cord (S340). After retracting the needle, the needle may be repositioned for repeated injection steps, as necessary (S350). Following completion of all injections, the needle is retracted and the XYZ manipulator (not shown) is removed from the stereotactic frame (not shown).

A dural closure is performed, followed by a laminary closure. The anatomical layers (muscle, subcuticle, skin) are then closed using resorbable materials (S360). Thus, the spinal pulsation-cancelation injection system 100 may be used in a human patient receiving direct spinal parenchymal injections of cells vectors, or drugs. As discussed above, the system 100 may eliminate any spinal cord pulsation effects that occur during a procedure of spinal cord injections in large animal species and in humans.

The following examples are intended to illustrate but not limit the invention.

### Example 1

A 30G non-coring needle built into the magnetic spinal-pulsation-cancellation system will be used to deliver test/therapeutic materials to the spinal cord of a subject. The spinal-pulsation cancellation system will be attached to a solid circular stainless steel bar with a needle holder (collectively called the catheter holder), which is connected to a sterilized XYZ manipulator (Stoelting; Cat.No: 51600). The XYZ manipulator will be mounted directly onto a stereotaxic frame. The injection needle will be interconnected with a horizontally oriented cell suspension coil constructed from Teflon Medical Micro Tubing (ID:0.01″; OD:0.02″; Scientific Commodities, INC; Ariz.), and positioned just above the injection needle. The diameter of the coil is 12 mm. Cell suspension is loaded into the coil manually by the surgeon using previously autoclaved cell-loaded Hamilton syringe (250 μl). The cell-loaded coil is then connected with CTS-filled Hamilton syringe (250 μl) mounted on a digital microinjector (Tritech Research; Model-MINJ-PD).

Set-up of Stereotactic Frame for Surgical Table—Fit the surgical table with the stereotactic frame, which supports the entire body of the patient. Attach the temperature-controlling pad to the stereotactic frame and cover with clean sheets.

Positioning the Patient—Position the patient on their prone surface with Thoracic Level 1 (T1) vertebra approximately at the first pair of stabilization bars. Follow standard of care to avoid compression points at the hip, abdomen, and chest.

Exposing Spinal Cord—Perform a standard posterior approach to target T1 to T10 followed by an “open door” laminoplasty, leaving the dura mater intact. Apply sterile saline to the operatory wound and apply sterile fields for protection during the next steps.

Installation of Stereotactic Arm, Stabilizing Bars, and Catheter Kit—Create openings in the sterile field to allow for the stereotactic arm to be attached to the stereotactic frame. Attach the stereotactic arm (XYZ manipulator) to the stereotactic frame. Attach the stabilizer bars. Aseptically isolate the sterile field openings. Install the catheter holder. Attach the catheter kit to the catheter holder by anchoring the upper and lower magnets of the pulse cancelling system into the holder.

Loading Catheter—Fill a 1 cc syringe with vehicle and fill the catheter tubing, checking for leakage at tubing unions. Using a 250 μL gas tight syringe (transfer syringe) with 18G needle, aspirate 240 μL of cell-suspension product. Retract the transfer syringe plunger 5 μL and attach the syringe to the catheter end within 5-15 seconds. Slowly load the contents of the transfer syringe into the catheter tubing, observing the air gap between the vehicle and cellular product. Remove the syringe after complete transfer of the cellular product. Close the catheter end with a sterile Luer cap provided in the catheter kit.

Micro-Injector Setup—The following describes the use of sterile methylene blue to act as a visual aid in the progression of the end of the cellular product throughout the catheter. The digital micro-injector is located on a clean table next to the patient and separated with sterile sheets. The injector plunge is completely inserted in the injector body at the end of run. A 3-way stop is attached to the injector body. Attach a 1 cc syringe with Methylene blue vial to the 3 way stop load port in the inverted position. Turn the 3 way stop to the load position. Set the micro-injector in reverse “R” to 0.2 μL/sec for a total of 5 μL actuation steps. Actuate the micro-injector in steps to fill with methylene blue until the 240 μL graduation. Do not actuate the injector to the end of run. Turn the 3-way stop to the off position. Feed the sterile capped catheter end through an opening in the sterile sheets. Remove the cap and attach the catheter to the 3 way stop on the micro-injector. Turn the 3-way stop to the “inject” position. Set the micro-injector in forward “F” to 0.2 μL/sec for a total of 5 μL actuation steps. Actuate the pump to allow the vehicle in the catheter to be completely displaced by the cellular product, observing the location and progression of the air gap separating the cellular product. Actuate the pump for 2 more steps to eliminate fluid containing the cellular product. During the above steps, the fluid from the catheter is eliminated on a sterile gauze that will be disposed. Set the digital microinjector settings to 1 μL/60 sec, 5 μL steps. Actuate the pump, observing the fluid at the needle tip. Abort the pump actuation and remove the liquid droplet from the needle by touching with sterile gauze. At this point the catheter is loaded with the cellular product and ready for dose delivery in the spinal cord parenchyma.

Administration of Cellular Product—Continue the surgical procedure by opening the dura mater, perform a longitudinal incision in the dura mater, avoiding damage to blood vessels. Position the needle of the Delivery Device System above the spinal cord median sulcus at the T1 level, and then move the needle laterally approximately ⅔ of the distance from the spinal cord median sulcus to the line of the emerging dorsal roots. The exact lateral needle displacement will be provided from the pre-operative MRI calculations. Lower the needle into the spinal cord parenchyma the entire distance to the plastic stop ring, avoiding damage to blood vessels under visual guidance. The length of the needle on the Delivery Device System will be selected from a stock of custom-length 30G non-coring needles based on the pre-operative MRI calculations for the depth of needle insertion to target the ventral horn of each patient. Confirm the placement of the injection needle and confirm normal electrophysiological responses with intraoperative (electrophysiological or neurophysiological) monitoring (IOM). Activate the digital micro-injector, which is set at 1 μL/60 seconds for a total of 5 μL. Following injection of 5 μL of MotorGraft Dose, deactivate the digital microinjector and leave the needle in place for 2 minutes before retracting and positioning to the next injection site. After retracting the needle, reposition the needle at the spinal cord median sulcus and move the needle laterally to the other side using the same distance as prior, and repeat the injection steps. Following completion of injections (bilateral) at T1, repeat for T2-T8 with the positioning specific to the respective level. Monitor the progression of the methylene blue solution throughout the surgical procedure. If the solution reaches the lower ⅓ portion of the catheter coil, the catheter assembly must be replaced from a new kit and re-filled with test article as described above. Following completion of all injections (bilateral from T1-T8), retract the Delivery Device needle and remove the stereotactic arm from the stereotactic frame.

Surgical Wound Closure—Perform dural closure and augment the closure with the fibrin glue Tisseel. Perform a laminary closure. Suture and wound close the anatomical layers (muscle, subcuticle, skin) using resorbable material.

Cleaning and Sterilization—Discard the single use components (catheter kit). Aseptically clean all other parts and sterilize.

Although the invention has been described with reference to the above example, it will be understood that modifications and variations are encompassed within the spirit and scope of the invention. Accordingly, the invention is limited only by the following claims.

What is claimed is:

1. A spinal cord pulsation-cancelation injection device comprising:
(a) a frame having an elongated body and a plurality of holders extending therefrom;
(b) a plurality of first magnets, each being fixedly attached to a holder;
(c) a tube having a first end and a second end, the tube being slidingly disposed within through-holes disposed in each holder and in each first magnet;
(d) a plurality of second magnets fixedly attached to an exterior surface of the tube; and
(e) a needle fixedly attached to the first end of the tube.

2. The spinal cord pulsation-cancelation injection device according to claim 1, wherein each of the first magnets and the each of the second magnets are disposed such that a north pole of one first magnet faces a north pole of one second magnet or a south pole of one first magnet faces a south pole of one second magnet, thereby providing a magnetic repulsive force upon which the tube floats.

3. The spinal cord pulsation-cancelation injection device according to claim 1, wherein the frame comprises two holders, each having attached thereto a first magnet, and a single second magnet is fixedly attached to the tube.

4. The spinal cord pulsation-cancelation injection device according to claim 1, wherein the frame comprises two holders, each having attached thereto a first magnet, and two second magnets are fixedly attached to the tube.

5. The spinal cord pulsation-cancelation injection device according to claim 1, further comprising a stop ring fixedly attached to the first end of the tube or an area near the first end of the tube.

6. The spinal cord pulsation-cancelation injection device according to claim 1, further comprising a stop ring fixedly attached to the second end of the tube or an area near the second end of the tube.

7. The spinal cord pulsation-cancelation injection device according to claim 1, wherein the frame is made from stainless steel.

8. The spinal cord pulsation-cancelation injection device according to claim 1, wherein the needle is from about 27 to about 32 gauge.

9. The spinal cord pulsation-cancelation injection device according to claim 1, further comprising tubing removably attached to the second end of the tube and configured for supplying a substrate to the needle.

10. A spinal cord pulsation-cancelation injection system comprising:
(a) the spinal cord pulsation-cancelation injection device according to claim 1; and
(b) a reservoir in fluid communication with the needle, the reservoir containing a substrate to be administered to a subject.

11. The system according to claim 10, further comprising a digital microinjector configured to control flow of the substrate through the needle.

12. The system according to claim 10, wherein the substrate is selected from the group consisting of cells, drugs, viruses, plasmids, and growth factors.

13. The system according to claim 10, wherein the frame comprises two holders, each having attached thereto a first magnet, and a single second magnet is fixedly attached to the tube.

14. The system according to claim 10, wherein the frame comprises two holders, each having attached thereto a first magnet, and two second magnets are fixedly attached to the tube.

15. A method of compensating for spinal cord pulsation during administration of a substrate to a spinal cord of a subject comprising:
(a) positioning the spinal cord pulsation-cancelation injection system according to claim 10 over the spinal cord of the subject;
(b) lowering the needle into the spinal cord; and
(c) delivering a dose of the substrate to the spinal cord,
wherein the needle and tube of the device float due to magnetic repulsive forces within the device, thereby compensating for spinal cord pulsation.

16. The method of claim 15, further comprising repeating each of the steps at multiple sites along the spinal cord.

17. The method of claim 15, wherein the step of delivering comprises activating a digital microinjector configured to control flow of the substrate through the needle.

18. The method of claim 15, wherein the step of lowering the needle comprises inserting the needle into the spinal parenchyma until a needle stop ring that is fixedly attached to the needle contacts the subject.
